(19) United States
(12) Patent Application Publication
(10) Pub. No.: US 2015/0297647 A1
(43) Pub. Date: Oct. 22, 2015

(54) ENTEROCOCCUS FAECIUM EF08 STRAIN, A USE, A FEED ADDITIVE AND A FEED THEREOF

(71) Applicant: SYNBIO TECH INC., Kaohsiung (TW)

(72) Inventors: Ting-Yu LEE, Kaohsiung (TW); Chih-Hsuan WANG, Kaohsiung (TW); Jin-Seng LIN, Kaohsiung (TW); Bi YU, Kaohsiung (TW); Tien-Ken HSU, Kaohsiung (TW); Lian-Chwan YANG, Kaohsiung (TW)

(21) Appl. No.: 14/689,698

(22) Filed: Apr. 17, 2015

(30) Foreign Application Priority Data
Apr. 17, 2014 (TW) 103114064
Aug. 14, 2014 (CN) 201410401691.6

(51) Int. Cl. A61K 35/744; A23K 1/00; A23K 1/17; A61K 35/747

(52) U.S. Cl. CPC A61K 35/744; A61K 35/747; A23K 1/009; A23K 1/17; A23Y 2220/67; A23Y 2220/03

(57) ABSTRACT

An Enterococcus faecium EF08 strain for manufacturing a feed additive is disclosed. The EF08 strain is deposited at Bioresource Collection and Research Center of Taiwan with deposit number BCRC 910525. Also, the EF08 strain is deposited at CGMCC-China General Microbiological Culture Collection with deposit number CGMCC 5549. Moreover, the EF08 strain has a 16S rDNA sequence as set forth as SEQ ID NO: 1.

### BACKGROUND OF THE INVENTION

1. Field of the Invention

The present invention generally relates to an Enterococcus faecium strain and, more particularly, to an Enterococcus faecium EF08 strain (EF08 strain) used as a feed additive. The present invention further relates to a plurality of uses of the EF08 strain, a feed additive including the EF08 strain and a feed including the EF08 strain.

2. Description of the Related Art

Terrestrial economic animals including livestock and poultry, such as pigs, cows, sheep, deer, chickens, ducks, and geese, can not only produce meat, milk, eggs, fur or other primary livestock products, but also become food or commodity derivatives via cooking or processing.

Low-dose antibiotics are usually incorporated into the feed during breeding the terrestrial economic animals. The incorporation of the low-dose antibiotics decreases microorganism level in the intestinal tracts, preventing the nutrients from consumption by the microorganisms. As such, the nutrients can be effectively converted into the primary livestock products, improving economic value of the terrestrial economic animals.

However, the overuse and misuse of antibiotics cause antibiotics residues in food of animal origin, affecting human heath, as well as triggers appearance of drug-resistant microorganisms in the intestinal tracts or even in the environment. Therefore, the use of the low-dose antibiotics is banned in European Union from 2006.

In light of this, it is necessary to provide a feed additive to replace the abovementioned low-dose antibiotics.

### SUMMARY OF THE INVENTION

It is therefore the objective of this invention to provide an EF08 strain. The EF08 strain improves intestinal microflora, enhancing immunity status of terrestrial economic animals.

It is another objective of this invention to provide a feed additive including the EF08 strain. By the incorporation of the feed additive including the EF08 strain, the terrestrial economic animals can effectively convert the nutrients into the primary livestock products, improving the economic value of the terrestrial economic animals.

A first embodiment of the invention discloses an Enterococcus faecium EF08 strain. The EF08 strain is deposited at Bioresource Collection and Research Center of Taiwan with deposit number BCRC 910525. The EF08 strain is also deposited at CGMCC-China General Microbiological Culture Collection with deposit number CGMCC 5549. Moreover, the EF08 strain has a 16S rDNA sequence as set forth as SEQ ID NO: 1.

A second embodiment of the invention discloses a use of the EF08 strain for manufacturing a feed additive for terrestrial economic animals.

The EF08 strain is incorporated into a feed for the terrestrial economic animals at dosages of 10⁴-10⁸ CFU/per gram of the feed.

A third embodiment of the invention discloses another use of the EF08 strain for enhancing secretion of IL-10, TNF-α, IL-12p40 and IFN-γ.

A fourth embodiment of the invention discloses yet another use of the EF08 strain for suppressing growth of E. coli and S. typhimurium.

A fifth embodiment of the invention discloses a feed additive including the EF08 strain at concentrations of 10⁸-10¹² CFU/per gram of the feed additive.

The feed additive further includes a Lactobacillus strain selected from a group consisting of a Lactobacillus plantarum LP28 strain and a Lactobacillus acidophilus LASW strain. The LP28 strain is deposited at CGMCC-China General Microbiological Culture Collection with deposit number CGMCC 3346. The LASW strain is deposited at CGMCC-China General Microbiological Culture Collection with deposit number CGMCC M204083.

The feed additive includes the Lactobacillus strain at concentrations of 10⁸-10¹² CFU/per gram of the feed additive.

The feed additive includes the LP28 strain and the LASW strain at concentrations of 10⁸-10¹² CFU/per gram of the feed additive and 10⁸-10¹¹ CFU/per gram of the feed additive, respectively.

A sixth embodiment of the invention discloses a feed including the EF08 strain at dosages of 10⁴-10⁸ CFU/per gram of the feed.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a bar chart showing acid tolerance of the EF08 strain.

FIG. 2 depicts a bar chart showing bile salts tolerance of the EF08 strain.

FIG. 3 depicts a bar chart showing the EF08 strain inhibits growth of pathogenic bacteria.

FIG. 4a depicts a bar chart showing the EF08 strain improves secretion of IL-10.

FIG. 4b depicts a bar chart showing the EF08 strain improves secretion of TNF-α.

FIG. 4c depicts a bar chart showing the EF08 strain improves secretion of IL-12p40.

FIG. 4d depicts a bar chart showing the EF08 strain improves secretion of IFN-γ secretion.

FIG. 5 depicts a bar chart showing the feed additive decreases fecal ammonia level of the weaned piglets.

FIG. 6 depicts a bar chart showing the feed additive decreases fecal pH value of the weaned piglets.

FIG. 7a depicts a bar chart showing the feed additive increases fecal lactic acid bacteria level of the weaned piglets.

FIG. 7b depicts a bar chart showing the feed additive decreases fecal E. coli level of the weaned piglet.

FIG. 7c depicts a bar chart showing the feed additive improves fecal microflora of the weaned piglets.

FIG. 8 depicts a bar chart showing the feed additive increases fecal IgA level of the weaned piglets.

FIG. 9a depicts a bar chart showing an increase of fecal lactobacilli level of the suckling piglets when sows are fed the feed additive during pregnancy and lactation period.

FIG. 9b depicts a bar chart showing a decrease of fecal E. coli level of the suckling piglets when sows are fed the feed additive during pregnancy and lactation period.

FIG. 9c depicts a bar chart showing an improvement of fecal microflora of the suckling piglets when sows are fed the feed additive during pregnancy and lactation period.

FIG. 10 depicts a bar chart showing an increase of fecal IgA level of the suckling piglets when sows are fed the feed additive during pregnancy and lactation period.

FIG. 11a depicts a line graph showing the feed additive increases egg production rate of layers during early laying period.

FIG. 11b depicts a line graph showing the feed additive increases average daily egg production of layers during early laying period.

FIG. 12a depicts a line graph showing the feed additive increases egg production rate of layers during peak laying period.

FIG. 12b depicts a line graph showing the feed additive increases average daily egg production of layers during peak laying period.

FIG. 13a depicts a line graph showing the feed additive increases egg production rate of layers during late laying period.

FIG. 13b depicts a line graph showing the feed additive increases average daily egg production of layers during late laying period.

FIG. 14 depicts a line graph showing the feed additive moderates titer decrease of anti-ND antibody of layers.

In the various figures of the drawings, the same numerals designate the same or similar parts. Furthermore, when the term “first”, “second”, “third”, “fourth”, “inner”, “outer” “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

An Enterococcus faecium EF08 strain according to the present invention is isolated from the feces of a healthy adult. The EF08 strain can decrease intestinal pH value, improve intestinal microflora and promote peripheral blood cells to secrete several cytokines.

The EF08 strain is isolated by a method including an isolation step (a), a characterization step (b) and a preservation step (c).

In the isolation step (a), a lactic acid bacteria strain is isolated from the feces of a healthy adult. Specifically, the feces of the healthy adult is inoculated in MRS media (TABLE 1) in a volumetric ratio of 1:50, and cultured for 20 hours in 37° C. The culture is then serial diluted and spread on MRS solid media (MRS media shown in TABLE 1 with 1.5% agar) to isolate the lactic acid bacteria strain which grow on the MRS solid media.

TABLE 1

| | Concentration (g/L) |
|---|---|
| Peptone | 10 |
| Beef Extract | 10 |
| Yeast Extract | 5 |
| Dextrose | 20 |
| Ammonium Citrate | 5 |
| MgSO₄ | 0.1 |
| MnSO₄ | 0.05 |
| K₂HPO₄ | 2 |
| Sodium Acetate | 5 |
| Tween 80 | 1 |
| | Water to 1 L |

In the characterization step (b), the isolated lactic acid bacteria strain is characterized by physical morphology, physiological property and molecular characteristics, such as gram staining, determination of enzyme activity and 16S rDNA sequencing thereof. Specifically, the isolated lactic acid bacteria strain belongs to Gram-positive bacteria with a 16S rDNA partial sequence as set forth as SEQ ID NO: 1. Moreover, the percentage of homology of the 16s rDNA partial sequence of the isolated lactic acid bacteria strain reaches up to 100% with Enterococcus faecium according to the NCBI/BLAST database. Thus, the isolated lactic acid bacteria strain belongs to Enterococcus faecium and therefore is named EF08. The EF08 strain according to the present invention is deposited at both Bioresource Collection and Research Center of Taiwan (deposit number: BCRC 910525) and CGMCC-China General Microbiological Culture Collection (deposit number: CGMCC 5549).

In the preservation step (c), for preservation, the EF08 strain according to the present invention is inoculated on the MRS solid media at 37° C. for 48 hours. A single colony is then transferred into the MRS media including an antifreeze (e.g. 30% glycerol) and preserved at −80° C.

To verify the potential of the EF08 strain as the feed additive, and to determine whether the feed additive including the EF08 strain improves the healthy status and economic values of the terrestrial economic animals, the following trials are carried out.

Trial (A). Acid Tolerance

The EF08 strain belonging to lactic acid bacteria is capable of secreting lactic acid, making the surrounding environment to a pH value of 3.2-4.5. However, the strong acidic environment (pH 2-3) in stomach due to gastric acid suppresses most lactic acid bacteria.

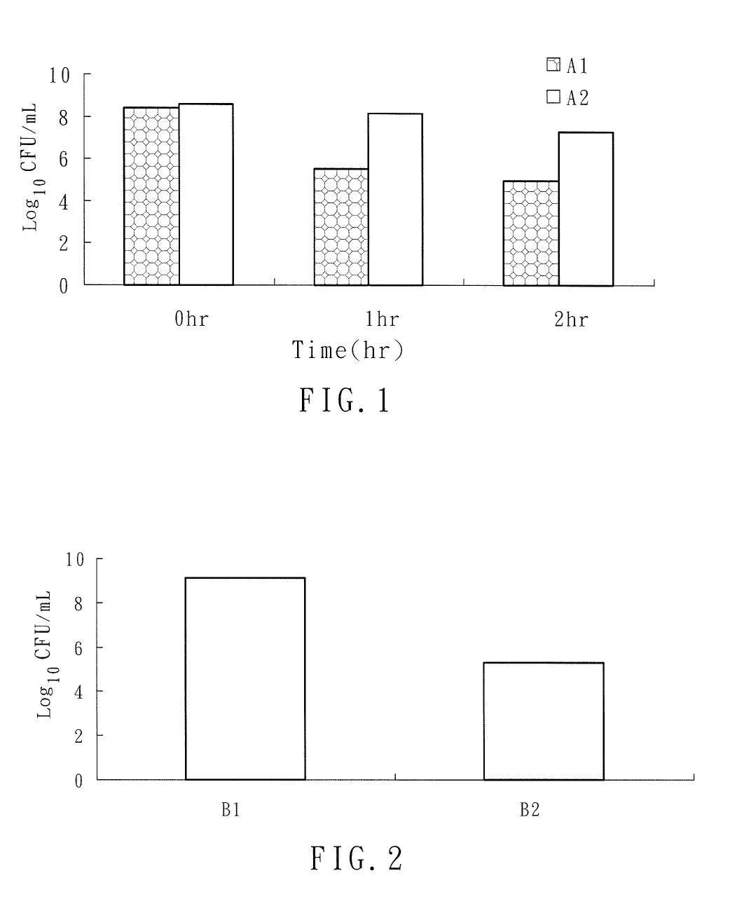

In trial (A), the EF08 cells are activated twice in the MRS media, followed by centrifugation at 5,000 rpm for 10 minutes to obtain the EF08 cells. The EF08 cells are then resuspended in 10-fold volume of sterile PBS buffer shown in TABLE 2 in a final concentration of 10⁹ CFU/mL and incubated for 0, 1 or 2 hours (37° C., 10 rpm). Finally, the treated EF08 cells are diluted, spread on the MRS solid media and cultured at 37° C. for 48-72 hours. The numbers of colonies are counted and recorded in FIG. 1.

TABLE 2

| Groups | Treatment |
|---|---|
| A1 | PBS (pH 2) |
| A2 | PBS (pH 3) |

Referring to FIG. 1, the longer the EF08 cells are in the low-pH PBS buffer, the fewer the EF08 cells survive. However, 10⁵ CFU/mL of the EF08 cells still survive after 2-hour treatment of the PBS buffer of group A1 (pH 2) and 10⁷ CFU/mL of the EF08 cells still survive after 2-hour treatment of the PBS buffer of group A2 (pH 3), indicating that the EF08 strain survives in the low-pH environment. Accordingly, the EF08 strain is capable of passing through gastric acid in the stomach, arriving the intestinal tract where the EF08 strain functions to improve the health of the host.

Trial (B). Bile Salts Tolerance

Bile salts, secreted by liver, are surfactants existing in the intestinal tract. Bile salts affect cell membrane permeability, resulting in the death of bacteria in the intestinal tract.

In trial (B), the EF08 cells are activated twice in the MRS media, followed by centrifugation at 5,000 rpm for 10 minutes to obtain the EF08 cells. The EF08 cells are then resuspended in the MRS media shown in TABLE 3 in a final concentration of 10⁹ CFU/mL and incubated for 16 hours (37° C.). The numbers of colonies are counted and recorded in FIG. 2.

TABLE 3

| Groups | Treatment |
|---|---|
| B1 | MRS media |
| B2 | MRS media with 0.3% bile salts |
| | (Oxgall) |

Referring to FIG. 2, after treatment with MRS media of group B2 (with 0.3% bile salts), more than 10⁵ CFU/mL of the EF08 cells survive, indicating that the EF08 strain survives in the environment containing bile salts. Accordingly, the EF08 strain is able to survive for long periods in the intestinal tract where the EF08 strain functions to improve the health of the host.

Trial (C). Adhesion to Epithelial Cells

Caco-2 cells (colonic epithelial cell, model of intestinal epithelial cells, group C1) and crop mucosa epithelial cells (epithelial cell of the avian digestive tract, group C2) are used in trial (C). Specifically, the Caco-2 cells are purchased from Bioresource Collection and Research Center of Taiwan (BCRC 910525) and activated in DMEM media containing 10% FBS for several times. The Caco-2 cells are then subcultured into the 24-well plate for 21 days. The crop mucosa epithelial cells are obtained from crops of chickens. In detail, the crops are dissected and washed using sterile PBS (pH 7.2). The mucosa epithelial cells are collected by scraping with microslides. After precipitation, the epithelial cells are diluted with the sterile PBS to a final concentration of 10⁴-10⁵ cells/mL.

The EF08 cells are collected by centrifugation at 5,000 rpm for 10 minutes, followed by resuspending in PBS. 100 μL of the collected EF08 cells (10⁹ CFU/mL) are co-cultured with Caco-2 cells (group C1) or crop mucosa epithelial cells (group C2) for 2 hours, respectively. Subsequently, the co-cultured EF08 cells are washed and lysed by 1% Triton-X. Finally, the numbers of colonies are counted using the MRS solid media, and cell numbers of the EF08 cells adhesive to Caco-2 cells (group C1) or crop mucosa epithelial cells (group C2) are calculated and recorded in TABLE 4.

TABLE 4

| | | Bacterial cell number |
|---|---|---|
| | | adhesive to epithelial |
| Groups | Cells | cells (CFU/cell) |
| C1 | Caco-2 | 335 |
| C2 | crop mucosa epithelial | 422 |
| | cells | |

Referring to TABLE 4, the EF08 cells adhere to epithelial cells of group C1 (Caco-2 cells) and group C2 (crop mucosa epithelial cells). That is, the EF08 strain is capable of adhering to either intestinal epithelial cells or epithelial cells of the avian digestive tract, and therefore can function either in the intestinal tract or in the avian digestive tract.

Trial (D). Growth Inhibition of Pathogenic Bacteria

S. typhimurium (No. STCC 510177, SynbioTech Inc.) and E. coli (No. STCC 510178, SynbioTech Inc.) are used as the model pathogenic bacteria in trial (D). The pathogenic bacterial cells are cultured overnight in NB media shown in TABLE 5 (in a final concentration of 10⁹ CFU/mL). 15 mL of sterile nutrient agar mixed with the cultured pathogenic bacterial cells is poured into a petri dish. After solidification, solid media with wells (φ9.25 mm) used in trial (D) is therefore obtained.

| | TALBE 5 |
|---|---|
| | Concentration (g/L) |
| Peptic digest of animal tissue | 5 |
| Sodium Chloride | 5 |
| Beef Extract | 1.5 |
| Yeast Extract | 1.5 |
| | Water to 1 L |

The activated EF08 cells are cultured in the MRS media for 24 hours, followed by centrifugation at 5,000 rpm for 10 minutes to obtain the EF08 cells. The supernatants (100 μL) are then dispensed into the wells on the solid media at 4° C. for 2 hours. Finally, diameters of the inhibition zones are measured after 14-15 hours of incubation at 37° C. Moreover, lactic acid (2%) is used as the positive control (group D2) as shown in TABLE 6.

TABLE 6

| Groups | Treatment |
|---|---|
| D1 | EF08 |
| D2 | Lactic acid (2%) |

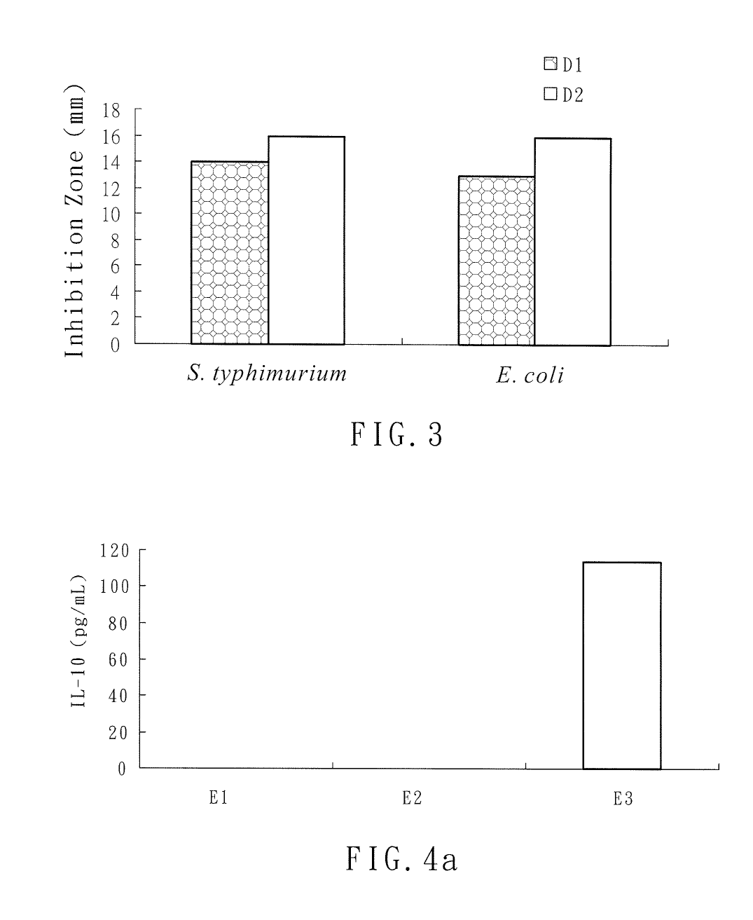

Referring to FIG. 3, treatment of group D1 (EF08) results in inhibition zones of about 16 mm against S. typhimurium and E. coli. Although compared to treatment of group D2 (2% lactic acid), the growth inhibition of pathogenic bacteria of treatment of group D1 (EF08) is worse, the EF08 strain still can inhibit growth of pathogenic bacteria, indicating that the EF08 strain shows potential to kill pathogenic bacteria in the intestinal tract.

Trial (E). Activity of Improving Secretion of Cytokines

In trial (E), peripheral blood mononuclear cells (PBMCs) are collected as following. Whole blood sample is first collected from brachial veins of chickens, and then mixed with Ficoll-Hypaque in a volumetric ratio of 1:1. The resultant is then centrifuged at 2,250 rpm for 30 minutes to obtain PBMCs. PBMCs are subsequently washed twice by PBS buffer and subcultured into 24-well plate in a final concentration of 1×10⁷ cells/well, followed by standing for 4 hours (37° C., 5% CO₂). Finally, PBMCs are co-cultured with the EF08 cells (1×10⁸ CFU/well) for 24 hours and cytokine secretion of PBMCs, especially secretion of IL-10, TNF-α, IL-12p40 and IFN-γ, is determined by ELISA.

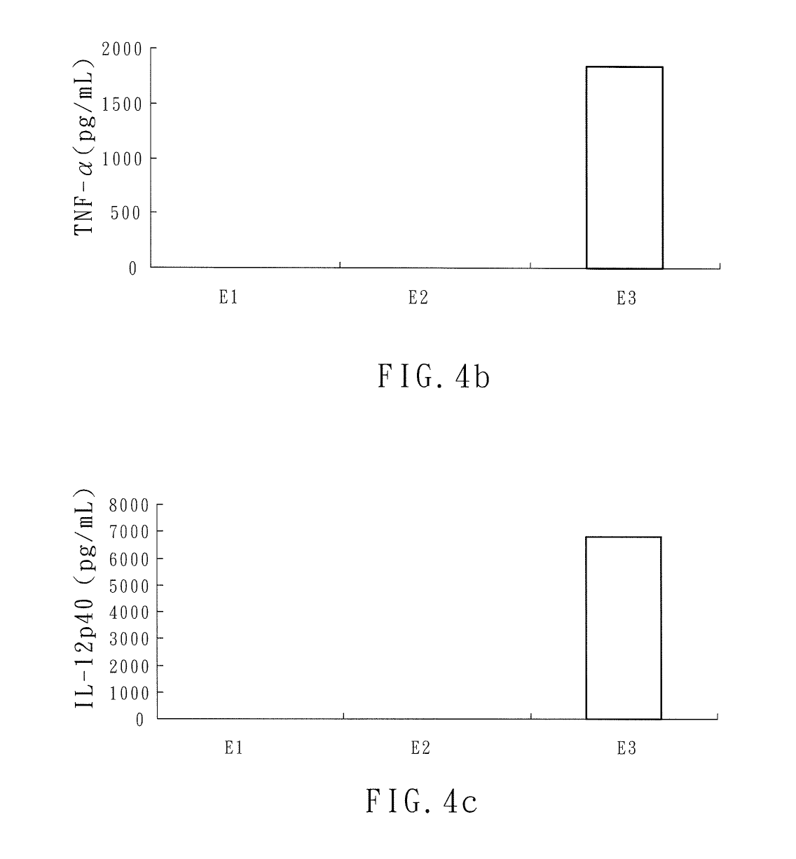

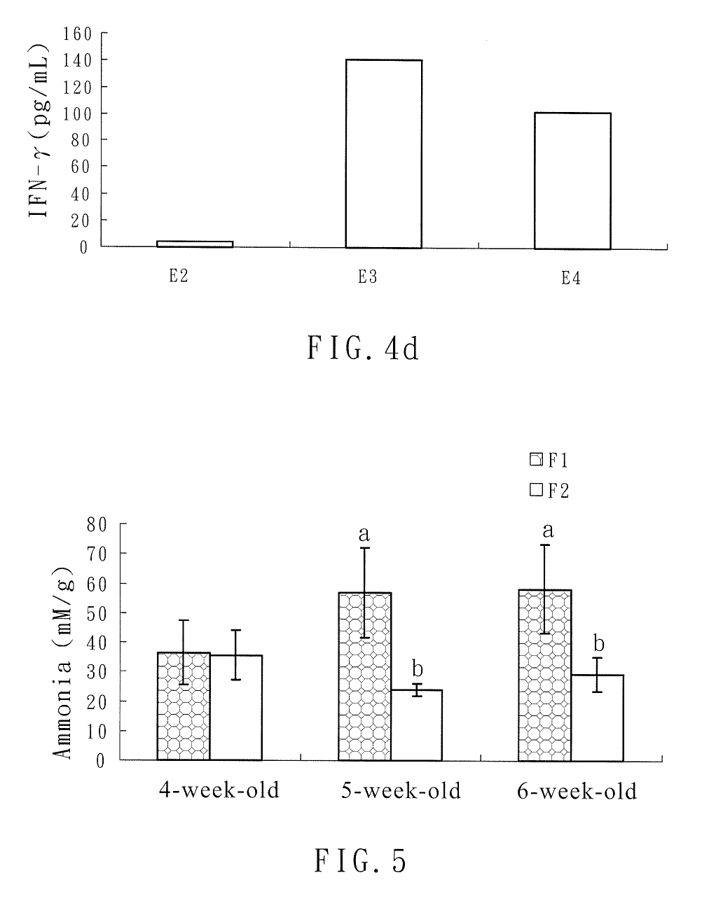

Referring to TABLE 7, group E1 is a blank control without treatment, group E2 is a negative control treated with PBS buffer, group E3 is treated with the EF08 cells, while group E4 is a positive control treated with concanavalin A (Con A). Secretion of IL-10, TNF-α, IL-12p40 or IFN-γ is shown in FIGS. 4a-4d, respectively.

TABLE 7

| Groups | Treatment |
|---|---|
| E1 | — |
| E2 | PBS |
| E3 | EF08 (1 × 10⁸ CFU/well) |
| E4 | Con A (5 μg/well) |

Referring to FIG. 4a, treatment of group E3 (EF08) effectively promotes secretion of IL-10. That is, the EF08 strain can effectively improve immune responses associated with IL-10, as an example, inhibition of activated macrophages and/or regulatory T cells secreting cytokines such as IFN-γ, IL-2, IL-3, TNF and GM-CSF, function of Th2 cells and/or mast cells, maturation of B cells and antibody production of B cells.

Referring to FIG. 4b, treatment of group E3 (EF08) effectively promotes secretion of TNF-α. That is, the EF08 strain can effectively improve immune responses associated with TNF-α, such as involvement of systematic inflammation and promotion of apoptosis by secreting downstream IL-1 and IL-6.

Referring to FIG. 4c, treatment of group E3 (EF08) effectively promotes secretion of IL-12p40, which is a subunit of IL-12 heterodimer. That is, the EF08 strain can effectively improve immune responses associated with IL-12, such as promotion of Th1 cells development, inhibition of Th2 cells development and improvement/maintenance of strength of Th 1-mediated cellular immune response.

Referring to FIG. 4d, treatment of group E3 (EF08) can effectively promote secretion of IFN-γ, which is secreted mainly by natural killer cells/natural killer T cells and plays an important role in both innate immunity and adaptive immunity. That is, the EF08 strain can effectively improve immune responses associated with IFN-γ, such as activation of macrophages, promoting immune mechanisms associated with macrophages.

In view of the foregoing, the EF08 strain according to the present invention has good tolerance to low pH and bile salts, being capable of passing through the stomach and arriving the intestinal tract where the EF08 strain can function to improve the health of host. The EF08 strain then adheres to the epithelial cells and inhibits growth of pathogenic bacteria in the intestinal tract. Moreover, the EF08 strain according to the present invention can effectively promote secretion of cytokines, such as IL-10, TNF-α, IL-12p40 and IFN-γ, enhancing the immune responses associated to the said cytokines. Therefore, the EF08 strain according to the present invention can be used as a feed additive, which is incorporated into the feed for the terrestrial economic animals, to effectively assure the terrestrial economic animals to have an improved immunity status and a better disease resistance ability.

Moreover, besides the EF08 strain, the feed additive according to the present invention preferably further includes a Lactobacillus strain selected from a group consisting of a Lactobacillus plantarum LP28 strain and a Lactobacillus acidophilus LASW strain. Specifically, the LP28 strain (been recited in Taiwan patent No. 201106957) is deposited at Bioresource Collection and Research Center of Taiwan (deposit number: BCRC 910435) and CGMCC-China General Microbiological Culture Collection (deposit number: CGMCC 3346). The LP28 strain has a 16S rDNA sequence as set forth as SEQ ID NO: 2. Moreover, the LASW strain (been recited in Taiwan patent No. 200708622 and China patent application No. 2005101054599) is deposited at Bioresource Collection and Research Center of Taiwan (deposit number: BCRC 910276) and CGMCC-China General Microbiological Culture Collection (deposit number: CGMCC M204083). The LASW strain has a 16S rDNA sequence as set forth as SEQ ID NO: 3.

Moreover, the feed additive according to the present invention includes effective amount of the EF08 strain, the LP28 strain and the LASW strain. As an example, the feed additive includes the EF08 strain at concentrations of 10⁸-10¹², the LP28 strain at concentrations of 10⁸-10¹² and the LASW strain at concentrations of 10⁸-10¹¹. Besides, the feed additive can further include an excipient selected from, but not limited to, a group consisting of maltodextrin, lactose and fermented soybean meals, which can be appreciated by a person having ordinary skill in the art. In addition, the feed additives used in the following trials are listed in TABLE 8.

TABLE 8

| | Trials | | | |
|---|---|---|---|---|
| Feed additive | (F) | (G) | (H) | (I) |
| EF08 (CFU/g | 10⁸ | 10¹² | 10¹⁰ | 10¹⁰ |
| of feed additive) | | | | |
| LP28 (CFU/g | 10¹² | 10⁸ | 10¹⁰ | 10¹² |
| of feed additive) | | | | |
| LASW | 10¹¹ | 10⁸ | 10¹⁰ | 10¹² |
| (CFU/g of | | | | |
| feed additive) | | | | |
| Maltodextrin | 20 | 20 | 20 | 20 |
| (g/100 g of | | | | |
| feed additive) | | | | |
| Lactose | 10 | 10 | 10 | 10 |
| (g/100 g of | | | | |
| feed additive) | | | | |
| Dose | 100 | 100 | 100 | 100 |
| (g/tonne of | | | | |
| feed) | | | | |
| Used for | Weaned piglets | Pregnant and | Layers | Broilers |
| | | lactating sows | | |

In addition, the feed additive according to the present invention is preferably stored in the cool, dry dark place to keep its activity.

To evaluate whether the feed additive including the EF08 strain can be used to improve the immunity status and disease resistance ability of the terrestrial economic animals, the EF08 strain, the LP28 strain and the LASW strain are incorporated into the feed as shown in TABLE 8. The resultant mixture is then fed to the terrestrial economic animals (e.g. as the weaned piglets in trial (F), the pregnant sows before labor in trial (G), the lactating sows after labor in trial (G), the layers in trial (H) and the broilers in trial (I)). Immune status of the terrestrial economic animals and feed conversion ratio (FCR) of the terrestrial economic animals are shown as following.

Trial (F). Effects on Weaned Piglets

In trial (F), the feed additive shown in TABLE 8 is incorporated into the feed, and used to feed the weaned piglets (4-week-old piglets) for 2 weeks.

Referring to TABLE 9, the weaned piglets with heavier average weight are used as a control group (group F1, fed with the commercial feed), and the weaned piglets with lighter average weight are used as an experimental group (group F2, fed with the commercial feed including the feed additive). The weaned piglets were fed ad libitum. The growth range is computed as (average weight of 6-week-old piglets−average weight of 4-week-old piglets)/(average weight of 4-week-old piglets). The feed conversion ratio is calculated as (total weight of feed intake)/(average weight of 6-week-old piglets−average weight of 4-week-old piglets).

TABLE 9

| | | | | | Feed |
|---|---|---|---|---|---|
| | | Average weight (kg) | | Growth | conversion |
| Groups | Feed | 4-week-old | 6-week-old | range | ratio |
| F1 | Commercial | 11.56 | 17.86 | 0.55 | 1.53 |
| | feed | | | | |
| F2 | Commercial | 9.89 | 16.33 | 0.68 | 1.41 |
| | feed with | | | | |
| | the feed | | | | |
| | additive | | | | |

Generally speaking, heaver piglets grow faster than the lighter ones. However, referring to TABLE 9, compared to the weaned piglets of group F1, the weaned piglets of group F2 show a greater growth range. Moreover, in respect to the feed conversion ratio, the weaned piglets of group F2 gain more weight compared to the weaned piglets of group F1 on the basis of same feed intake. It is clear that the feed additive according to the present invention effectively promotes the growth of the weaned piglets.

Referring to FIG. 5, fecal ammonia level in the weaned piglets of group F2 significantly decreases, indicating that the feed additive according to the present invention reduces bad odor of feces.

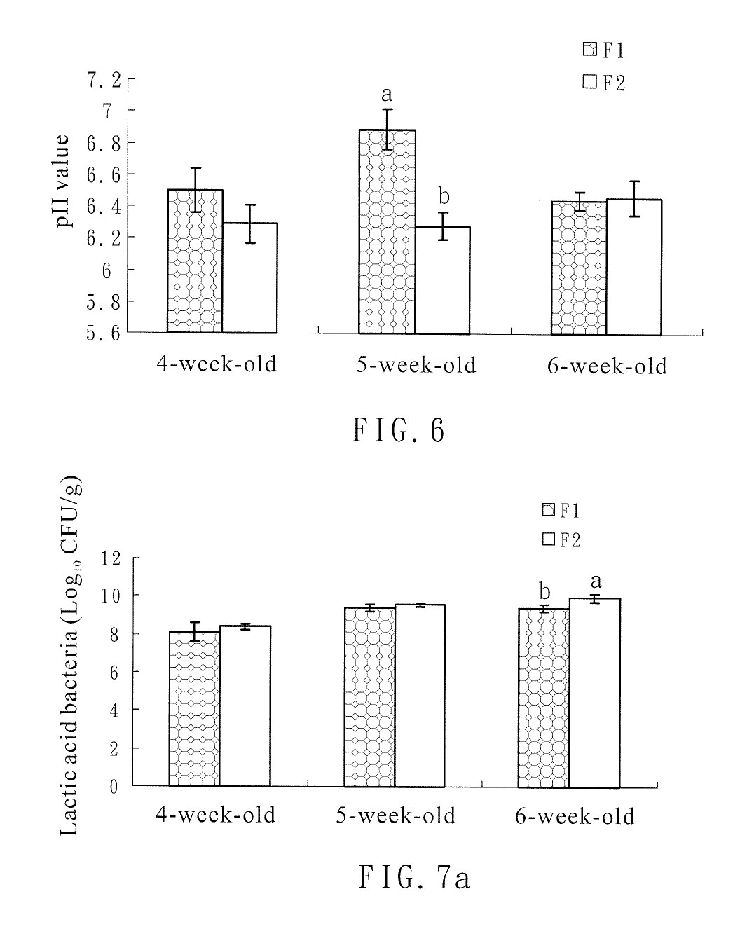

Referring to FIG. 6, piglets, with immature intestinal tract, have slight acidic feces due to the suckled milk provided by the lactating sows before weaning. After weaning, (e.g. one week), the pH value of the feces is about 7. However, after the development of intestinal tract is completed (e.g. two weeks after weaning), the feces again has a slight acidic pH. On the other hand, compared to the 5-week-old weaned piglets of group F1, the 5-week-old weaned piglets of group F2 have slight acidic feces, indicating that the 5-week-old weaned piglets of group F2 have a healthier intestinal tract.

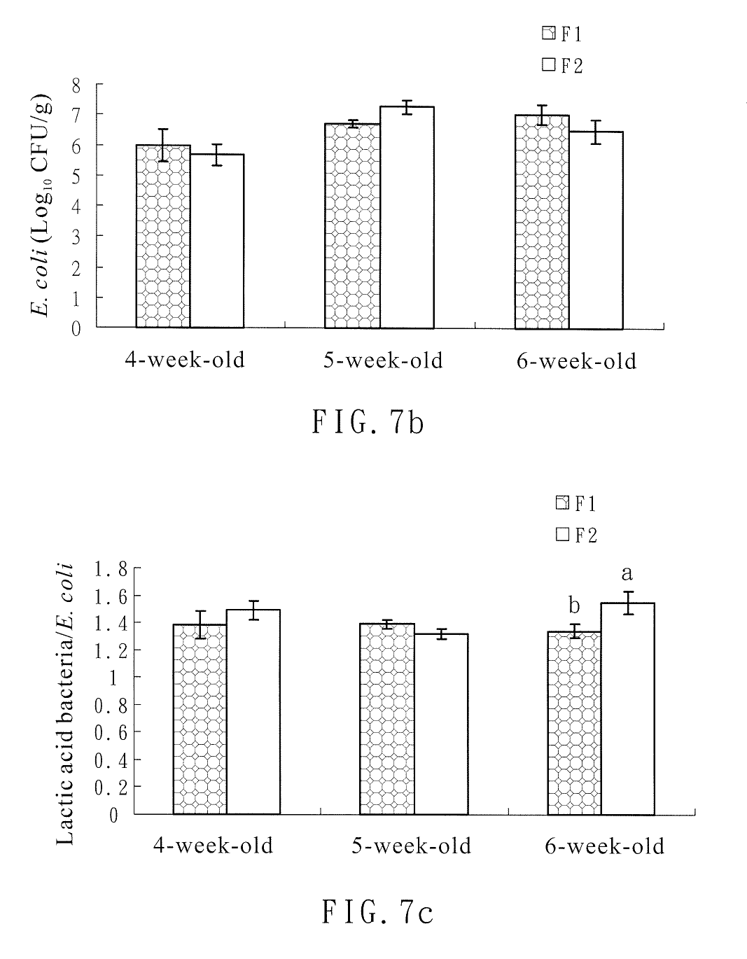

FIGS. 7a and 7b show good bacteria (lactic acid bacteria) and bed bacteria (E. coli) level in feces of the weaned piglets, respectively. Moreover, FIG. 7c shows the fecal microflora (lactic acid bacteria/E. coli), indicating health status of intestinal tract. The results show that incorporation of the feed additive according to the present invention not only increases the probiotic bacteria level, but also decreases the pathogenic bacteria level, regulating microflora in the feces of the weaned piglets. That is, incorporation of the feed additive is capable of improving healthy intestinal tract.

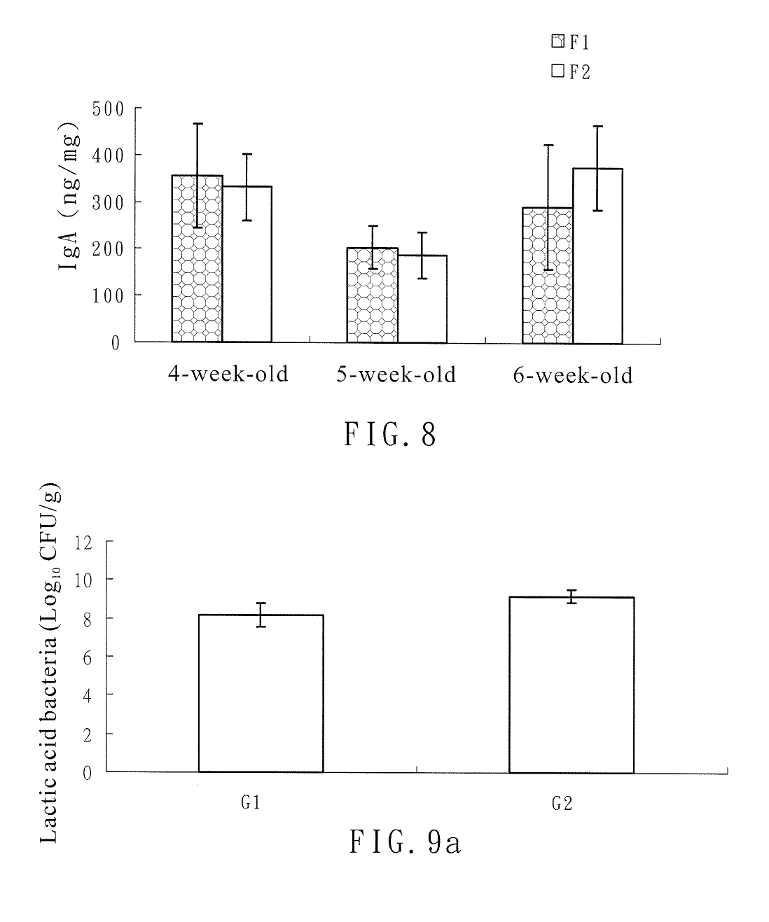

Referring to FIG. 8, we can find as high as 350 ng/mg of IgA in the feces of the F1 group 4-week-old weaned piglets, which immune system is still not well developed. Sow milk gives them the high IgA titer. After weaning, (e.g. one week), the IgA level of the feces drops to 200 ng/mg. However, after the completeness of immune system (e.g. two weeks after weaned), the IgA level of the feces again rises to nearly 300 ng/mg. In contrast, compared to the 6-week-old weaned piglets of group F1, the 6-week-old weaned piglets of group F2 have significantly higher IgA level. Therefore, by increasing the IgA level, the feed additive according to the present invention can effectively improve the immune status of the weaned piglet.

In view of the foregoing, the feed additive according to the present invention incorporated into commercial feeds for the weaned piglets can improve immunity status and disease resistance ability of the weaned piglets. Moreover, the feed additive can inhibit growth of pathogenic bacteria in the intestinal tract, preventing the nutrients from consumption by pathogenic bacteria in the intestinal tract. As such, the weaned piglets show an increased feed conversion ratio.

Trial (G). Effects on Pregnant and Lactating Sows

In trial (G), the feed additive shown in TABLE 8 is incorporated into the feed, and used to feed the pregnant sow having a due date on 2 weeks later during the pregnant and lactating period. The pregnant sow then farrows a newborn piglet and the newborn piglet suckles milk of the lactating sow for 2 weeks.

Referring to TABLE 10, in the control group (group G1), the sows are fed with commercial feed, while in the experimental group (group G2), the sows are fed with commercial feed including the feed additive according to the present invention. Moreover, the sows of both groups G1 and G2 are housed and fed ad libitum. The growth range is computed as (average weight of 2-week-old suckling piglets−average weight of 0-week-old suckling piglets)/(average weight of 0-week-old suckling piglets).

TABLE 10

| | | Average weight (kg) | | Growth |
|---|---|---|---|---|
| Groups | Feed | 0-week-old | 2-week-old | range |
| G1 | Commercial | 2.14 | 3.80 | 0.76 |
| | feed | | | |
| G2 | Commercial | 1.69 | 4.17 | 1.48 |
| | feed with the | | | |
| | feed additive | | | |

Referring to TABLE 10, compared to the suckling piglets of group G1, the suckling piglets of group G2 considered having a smaller growth range show a greater growth range. Notably, the length of pregnancy of sows is about 110-120 days, the fetal weights within the same litter diverse after 35 day of pregnancy. That is to say, when the pregnant sows start being fed by the feed with the feed additive, litters of piglets have already shown difference on weight, the incorporation of the feed additive could not affect the birth weight of piglets.

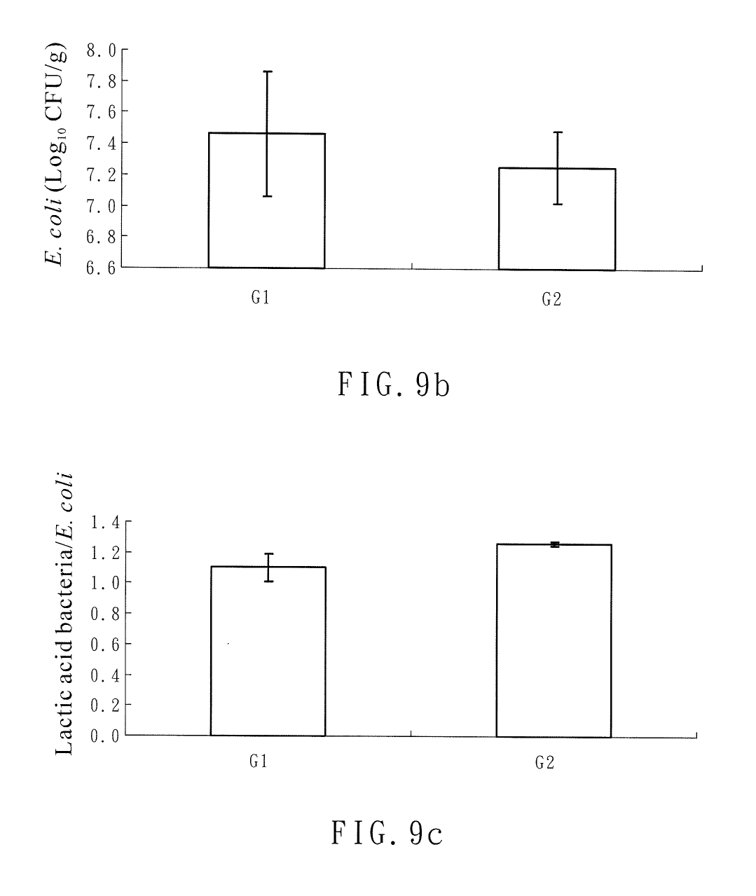

Referring to FIGS. 9a-9c, the incorporation of the feed additive increases the fecal lactic acid bacteria level, decreases the fecal pathogenic bacteria level and regulates fecal microflora of the suckling piglets, assuring the suckling piglets have a healthier intestinal tract.

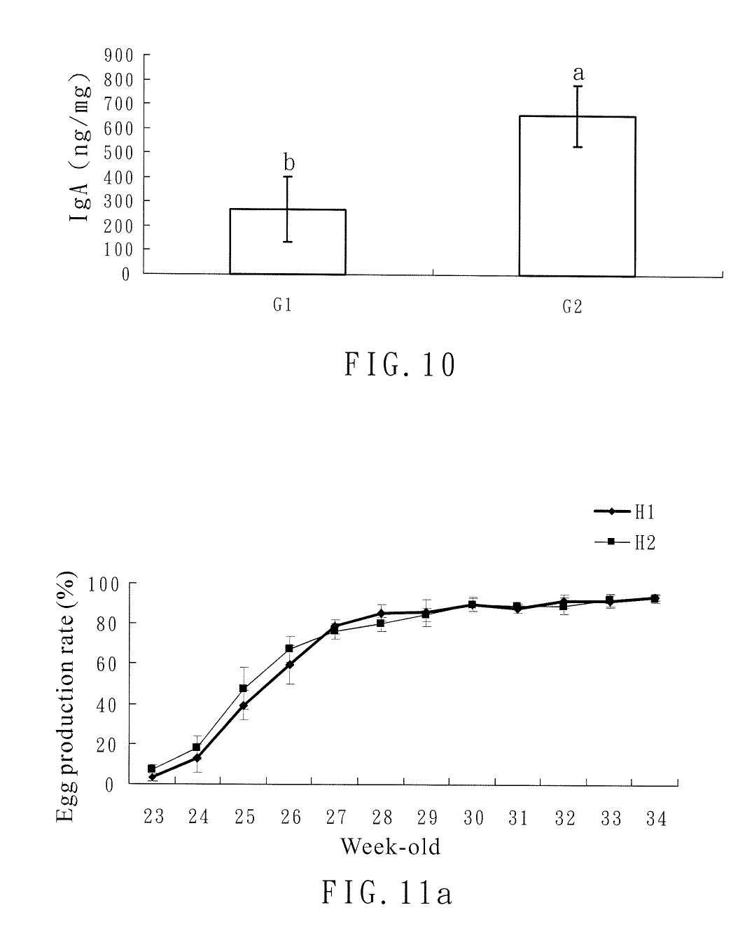

Referring to FIG. 10, the suckling piglets of group G1 have an fecal IgA level up to about 270 ng/mg while the suckling piglets of group G2 have an fecal IgA level of about 600 ng/mg, indicating the incorporation of the feed additive to the feed of sows during the pregnancy and lactation significantly increases the intestinal IgA level, improving immune ability of the suckling piglets.

In view of the foregoing, the feed additive according to the present invention can be incorporated into commercial feeds for pregnant and lactating sows. With such performance, the suckling piglets have an improved immunity status and a better disease resistance ability.

Trial (H). Effects on Layers

Referring to TABLE 11, 20-week-old layers are divided into group H1 (fed with the commercial feed) and group H2 (fed with the commercial feed with the feed additive according to the present invention). The feed continues from the layers are 20-week-old to 34-week-old, and from the layers are 43-week-old to 80-week-old.

TABLE 11

| Groups | Feed |
|---|---|
| H1 | Commercial feed |
| H2 | Commercial feed with the feed |
| | additive |

Generally, laying period of layers is divided into early laying period (from about 23-week old), peak laying period (about 50-week-old) and late laying period (after about 70-week-old). Specifically, in early laying period, only few layers lay eggs; in peak laying period, both egg production rate and average daily egg production arrive peak stage; and in late laying period, both egg production rate and average daily egg production decrease. In trial (H), egg production rate and average daily egg productions in the abovementioned laying periods are compared.

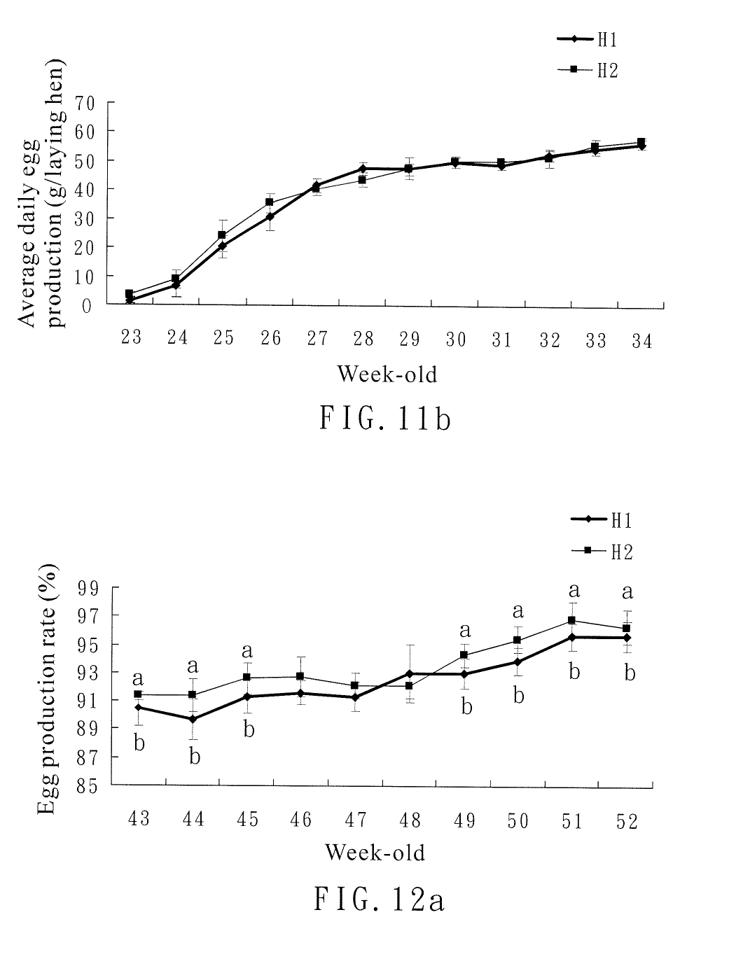

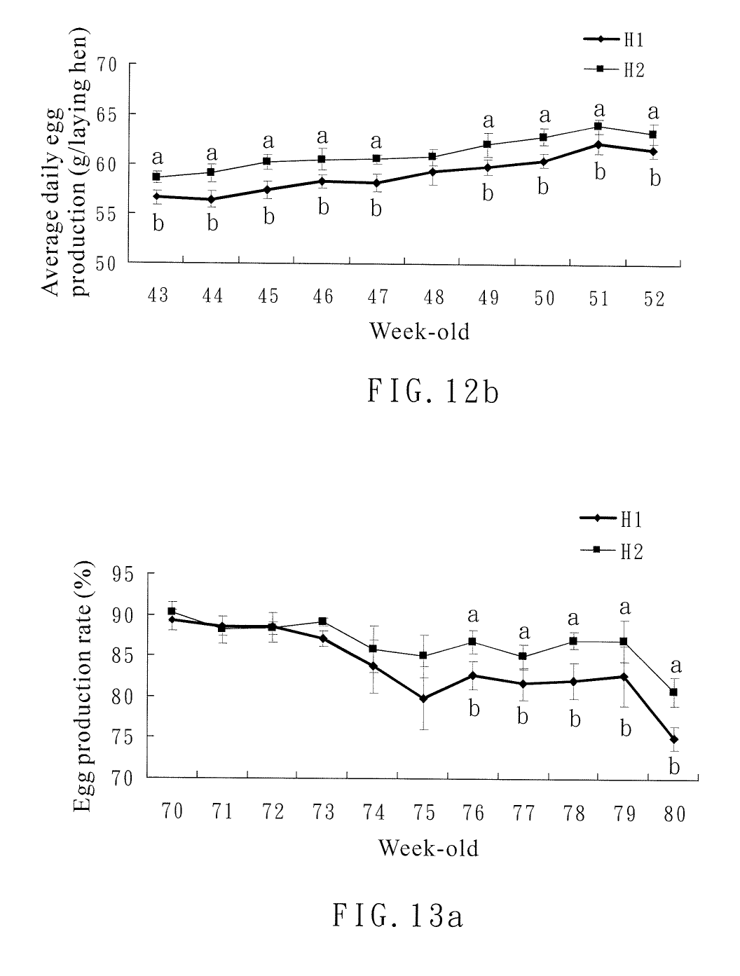

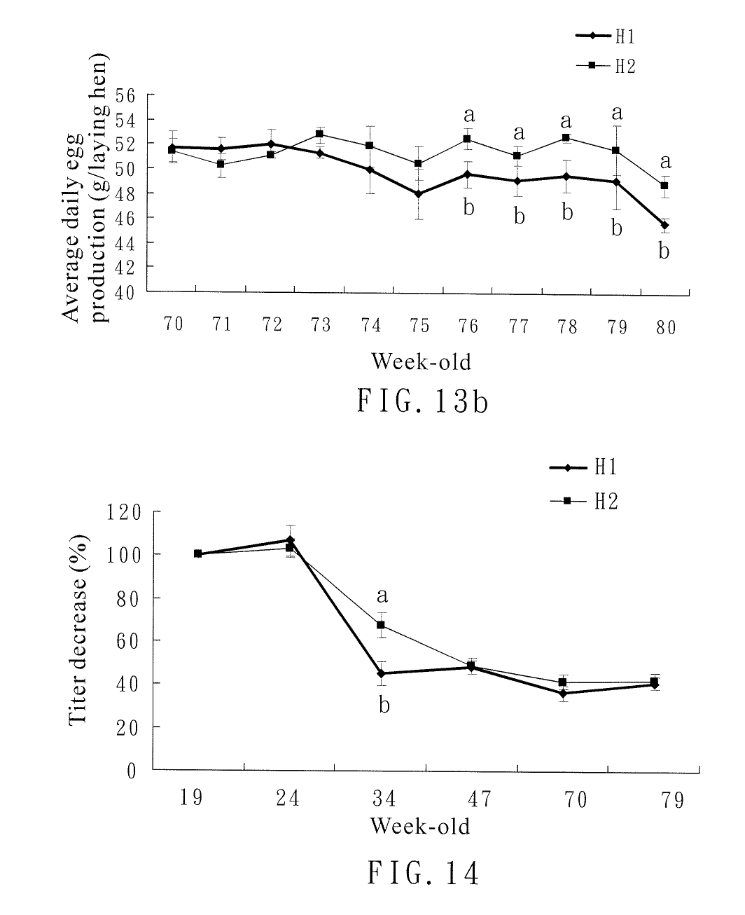

In early laying period, compared to layers of group H1 fed by commercial feeds, layers of group H2 fed by commercial feeds with feed additive show a higher egg production rate (shown in FIG. 11a) and a higher average daily egg production (shown in FIG. 11b) from week 23. Moreover, in peak laying period, layers of group H2 also show a higher egg production rate and a higher average daily egg production shown in FIGS. 12a and 12b. In addition, referring to FIGS. 13a and 13b, in late laying period, compared to layers of group H1, layers of group H2 show not only a higher egg production rate and a higher average daily egg production, but also a moderate downward tendency to lay eggs. That is, the incorporation of the feed additive according to the present invention prolongs laying period of layers, as well as increases average daily egg production.

Furthermore, titer decrease of antibody against Newcastle disease (anti-ND antibody) is measured. The vaccination program of Newcastle disease according to a certain farm is carried out. Whole blood samples are collected from brachial vein on week 19, week 24, week 34, week 47, week 70 and week 79. After clotting, the whole blood samples are centrifuged at 10,000 rpm for 5 minutes. The supernatants are stored at −80° C. for further test using IDEXX ELISA kit.

Referring to FIG. 14, compared to layers of group H1, layers of group H2 fed with commercial feeds containing feed additive shows a slighter titer decrease, indicating incorporation of the feed additive according to the present invention prolongs half-life of anti-ND antibody. As such, frequent and/or high dose vaccination can be prevented.

In view of the foregoing, the feed additive according to the present invention incorporated into commercial feeds for layers can not only improve intestinal microflora but also elevate cellular immunity and/or humoral immunity. On the other hand, the feed additive according to the present invention also shows effects on nutrients intake, improving egg production rate and average daily egg production.

Trial (I). Effects on Broilers

Referring to TABLE 12, broilers (between 1-day-old and 35-day-old) are divided into group I1 (fed with commercial feed), group I2 (fed with commercial feed containing the feed additive according to the present invention) and group I3 (fed with commercial feed containing low-dose antibiotics).

TABLE 12

| Groups | Feed |
|---|---|
| I1 | Commercial feed |
| I2 | Commercial feed with the feed |
| | additive |
| I3 | Commercial feed with low-dose |
| | antibiotics |

Feed conversion rate of broilers is calculated as (total weight of feed intake)/(increased weight) and recorded in TABLE 13.

TABLE 13

| | Feed conversion rate | | |
|---|---|---|---|
| | 1-day-old to | 21-day-old to | 1-day-old to |
| Groups | 21-day-old | 35-day-old | 35-day-old |
| I1 | 1.28 | 1.78 | 1.59 |
| I2 | 1.28 | 1.74 | 1.57 |
| I3 | 1.29 | 1.72 | 1.56 |

Referring to TABLE 13, compared to broilers of group I1, broilers of group I2, as well as broilers of group I3, shows a lower feed conversion rate, indicating the incorporation of the feed additive according to the present invention promotes growth status of broilers.

Intestinal pH and volatile fatty acid level of 35-day-old broilers of groups I1 to I3 are recorded in TABLE 14. As a result, compared to broilers of group I3, broilers of group I2 has a lower intestinal pH, indicating that the incorporation of the feed additive according to the present invention improves intestinal status of broilers, thereby preventing pathogenic bacteria from survival and proliferation in intestinal tract.

TABLE 14

| | |
|---|---|
| | Volatile fatty acid |
| pH value | (μmol/g) |

| Groups | Crop | Ileum | Cecum | Ileum | Cecum |
|---|---|---|---|---|---|
| I1 | 4.88 | 6.59 | 7.36 | 4.95 | 22.98 |
| I2 | 5.02 | 6.86 | 7.24 | 4.72 | 22.39 |
| I3 | 5.14 | 8.10 | 6.89 | 5.15 | 22.92 |

Intestinal probiotic bacteria (e.g. lactic acid bacteria) level and intestinal pathogenic bacteria (e.g. E. coli and Clostridium perfringens) level of 35-day-old broilers of groups I1 to I3 are recorded in TABLE 15. As a result, compared to broilers of group I1, broilers of group I2 show decreased intestinal pathogenic bacteria level and increased intestinal probiotic bacteria level, indicating that incorporation of the feed additive according to the present invention regulates intestinal microflora of broilers, that is, increases intestinal probiotic bacteria level and decreases intestinal pathogenic bacteria level.

TABLE 15

| | Intestinal microflora (Log₁₀ CFU/g) | | | | | | | | |
|---|---|---|---|---|---|---|---|---|---|
| | Lactic acid | | | | | | | | |
| | bacteria | | | E. coli | | | C. perfringens | | |
| | | Ile- | Ce- | | Ile- | Ce- | | Ile- | Ce- |
| Groups | Crop | um | cum | Crop | um | cum | Crop | um | cum |
| I1 | 7.80 | 7.93 | 8.27 | 6.68 | 7.61 | 8.72 | 3.92 | 3.73 | 3.63 |
| I2 | 8.78 | 8.30 | 8.36 | 6.48 | 6.96 | 8.28 | 3.29 | 3.01 | 3.29 |
| I3 | 8.17 | 7.44 | 8.29 | 6.80 | 7.27 | 7.97 | 3.72 | 4.18 | 3.75 |

In view of the foregoing, the feed additive according to the present invention incorporated into commercial feeds for broilers can not only decrease intestinal pH value, but also improve intestinal microflora. Such as, broilers will show improved immunity status and a better disease resistance ability. Moreover, the feed additive according to the present invention also shows effects on nutrients intake, increasing feed conversion rate of broilers.

In conclusion, the EF08 strain according to the present invention can pass through gastric acid in the stomach, arriving the intestinal tract and adhering to the intestinal epithelial cells. Moreover, the EF08 strain can then secret lactic acid, lowering the intestinal pH value. As such, the EF08 strain can not only regulate intestinal microflora but also promote secretion of cytokines (e.g. IL-10, TNF-α, IL-12p40 and IFN-γ), assuring the terrestrial economic animals have an improved immunity status and a better disease resistance ability.

Moreover, the feed additive according to the present invention can prevent the nutrients from consumption by pathogenic bacteria in the intestinal tract. With such performance, the terrestrial economic animals can effectively convert the nutrients into primary livestock products and therefore have an improved economic value.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. An Enterococcus faecium EF08 strain, deposited at Bioresource Collection and Research Center of Taiwan with deposit number BCRC 910525, deposited at CGMCC-China General Microbiological Culture Collection with deposit number CGMCC 5549, and with a 16S rDNA sequence as set forth as SEQ ID NO: 1.

2. A use of the EF08 strain described in claim 1, wherein the EF08 strain is used to manufacture a feed additive for terrestrial economic animals.

3. The use according to claim 2, wherein the EF08 strain is incorporated into a feed for the terrestrial economic animals at dosages of 10⁴-10⁸ CFU/per gram of the feed.

4. A use of the EF08 strain described in claim 1, wherein the EF08 strain is used to enhance secretion of IL-10, TNF-α, IL-12p40 and IFN-γ.

5. A use of the EF08 strain described in claim 1, wherein the EF08 strain is used to suppress growth of Escherichia coli and Salmonella typhimurium.

6. A feed additive comprising the EF08 strain described in claim 1, wherein the feed additive comprises the EF08 strain at concentrations of 10⁸-10¹² CFU/per gram of the feed additive.

7. The feed additive according to claim 6, wherein the feed additive further comprises a Lactobacillus strain selected from a group consisting of a Lactobacillus plantarum LP28 strain and a Lactobacillus acidophilus LASW strain, wherein the LP28 strain is deposited at CGMCC-China General Microbiological Culture Collection with deposit number CGMCC 3346, and wherein the LASW strain is deposited at CGMCC-China General Microbiological Culture Collection with deposit number CGMCC M204083.

8. The feed additive according to claim 7, wherein the feed additive comprises the Lactobacillus strain at concentrations of 10⁸-10¹² CFU/per gram of the feed additive.

9. The feed additive according to claim 8, wherein the feed additive comprises the LP28 strain at concentrations of 10⁸-10¹² CFU/per gram of the feed additive and the LASW strain at concentrations of 10⁸-10¹¹ CFU/per gram of the feed additive.

10. A feed comprising the EF08 strain described in claim 1, wherein the EF08 strain is at dosages of 10⁴-10⁸ CFU/per gram of the feed.


SEQUENCE LISTING (3 sequences)

SEQ ID NO: 1 - DNA, 1366 bp, Enterococcus faecium
ctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagag

SEQ ID NO: 2 - DNA, 880 bp, Lactobacillus plantarum
agcagccgcggtaatacgtaggtggcaagcgttgtccggatttattgggcgtaaagcgagcgcaggcggttttttaagtctgatgtgaaagctttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcctttcagtgctgcagctaacgcattaagcattccgcctggggagtacggccgcaaggctgaaactcaaaggaattgacgggggcccgcacaagcggtggagcatgtggtttaattggaagttacgcgaagaaccttaccaggtcttgacatactatgcaaatttaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacac

SEQ ID NO: 3 - DNA, 1011 bp, Lactobacillus acidophilus
ttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaaaaataagtctaatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatctgtagagatacggagttcccttcggggacgctaagacaggtggtgcatggctgtcgtcagctcgtgtcgtgagatgttgggttaagtcccgcaacgagcgcaacccttgttattagttgccagcattaagttgggcactctaatgagactgccggtgacaaaccggaggaaggtggggatgacgtcaagtcatcatgccccttatgacctgggctacacacgtgctacaatgggcagtacaacgagaagcaagcctgcgaaggcaagcgaatctctgaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttc